(19) United States
(12) Patent Application Publication
(10) Pub. No.: US 2020/0255438 A1
(43) Pub. Date: Aug. 13, 2020

(54) NOVEL SALTS

(71) Applicant: Biogen Inc., Cambridge, MA (US)

(72) Inventors: Gerard M. Giblin, Cambridge, MA (US); David T. MacPherson, Cambridge, MA (US); Michael Williams, Cambridge, MA (US); David R. Witty, Cambridge, MA (US); Julian Northen, Cambridge, MA (US); Kalyan Vasudevan, Cambridge, MA (US)

(21) Appl. No.: 16/651,577

(22) PCT Filed: Sep. 28, 2018
(86) PCT No.: PCT/US18/53520

§ 371 Date: Mar. 27, 2020

Related U.S. Application Data
(60) Provisional application No. 62/564,744, filed on Sep. 28, 2017.

(51) Int. Cl. C07D 487/10

(52) U.S. Cl. CPC C07D 487/10; C07B 2200/13

(57) ABSTRACT

The invention relates to novel salts of 7-methyl-2-[4-methyl-6-[4-(trifluoromethyl)-phenyl]pyrimidin-2-yl]-1,7-diazaspiro[4.4]nonan-6-one, to compositions containing said salts and to the use of said salts in treating diseases and conditions mediated by modulation of voltage-gated sodium channels.

### RELATED APPLICATIONS

This application claims the benefit of U.S. Provisional Patent Application No. 62/564,744, filed on Sep. 28, 2017. The contents of this application are hereby incorporated by reference in their entirety.

### FIELD OF THE INVENTION

The invention relates to novel salts of 7-methyl-2-[4-methyl-6-[4-(trifluoromethyl)-phenyl]pyrimidin-2-yl]-1,7-diazaspiro[4.4]nonan-6-one, to compositions containing said salts and to the use of said salts in treating diseases and conditions mediated by modulation of voltage-gated sodium channels.

### BACKGROUND OF THE INVENTION

Voltage-gated sodium channels are responsible for the initial phase of the action potential, which is a wave of electrical depolarisation usually initiated at the soma of the neuron and propagated along the axon to the terminals. At the terminals, the action potential triggers the influx of calcium and the release of neurotransmitter. Drugs, such as lidocaine, that block voltage-gated sodium channels are used as local anaesthetics. Other sodium channel blockers, such as lamotrigine and carbamazepine are used to treat epilepsy. In the latter case, partial inhibition of voltage-gated sodium channels reduces neuronal excitability and reduces seizure propagation. In the case of local anaesthetics, regional block of sodium channels on sensory neurons prevents the conduction of painful stimuli. A key feature of these drugs is their state-dependent mechanism of action. The drugs are thought to stabilise an inactivated conformation of the channel that is adopted rapidly after the channel opens. This inactivated state provides a refractory period before the channel returns to its resting (closed) state ready to be reactivated. As a result, state-dependent sodium channel blockers inhibit the firing of neurons at high frequency, for example in response to painful stimuli, and will help to prevent repetitive firing during periods of prolonged neuronal depolarisation that might occur, for example, during a seizure. Action potentials triggered at lower frequencies, for example in the heart, will not be significantly affected by these drugs, although the safety margin differs in each case, since at high enough concentrations each of these drugs is capable of blocking the resting or open states of the channels.

The voltage-gated sodium channel family is made up of 9 subtypes, four of which are found in the brain, NaV1.1, 1.2, 1.3 and 1.6. Of the other subtypes, NaV1.4 is found only in skeletal muscle, NaV1.5 is specific to cardiac muscle, and NaV1.7, 1.8, and 1.9 are found predominantly in sensory neurons. The hypothesised binding site for state-dependent sodium channel blockers is the local anaesthetic (LA) binding site in the inner vestibule of the pore on transmembrane S6 of domain IV. Critical residues are located in a highly conserved region among the different subtypes, thus presenting a challenge for the design of new subtype selective drugs. Drugs such as lidocaine, lamotrigine and carbamazepine do not distinguish between the subtypes. However, selectivity can be achieved, and can be further enhanced functionally, as a result of the different frequencies at which the channels operate.

Drugs that block voltage-gated sodium channels in a state-dependent manner are also used in the treatment of bipolar disorder, either to reduce symptoms of mania or depression, or as mood stabilisers to prevent the emergence of mood episodes. Clinical and preclinical evidence also suggests that state-dependent sodium channel blockers may help to reduce the symptoms of schizophrenia. For example, lamotrigine has been shown to reduce symptoms of psychosis induced by ketamine in healthy human volunteers, and furthermore, studies in patients suggest that the drug can augment the antipsychotic efficacy of some atypical antipsychotic drugs, such as clozapine or olanzapine. It is hypothesised that efficacy in these psychiatric disorders may result in part from a reduction of excessive glutamate release. The reduction in glutamate release is thought to be a consequence of sodium channel inhibition in key brain areas, such as the frontal cortex. However, interaction with voltage-gated calcium channels may also contribute to the efficacy of these drugs.

WO 2013/175205 (Convergence Pharmaceuticals Limited) describes (2R,5S)-7-methyl-2-[4-methyl-6-[4-(trifluoromethyl)-phenyl]pyrimidin-2-yl]-1,7-diazaspiro[4.4]nonan-6-one hydrochloride, sulfuric acid salt and sulfuric acid salt hydrate which are claimed to be modulators of voltage-gated sodium channels. The object of the invention is to identify alternative salts of said compound which have advantageous properties.

### SUMMARY OF THE INVENTION

According to a first aspect, the invention provides a compound of formula (I) which is a pharmaceutically acceptable salt of 7-methyl-2-[4-methyl-6-[4-(trifluoromethyl)-phenyl]pyrimidin-2-yl]-1,7-diazaspiro[4.4]nonan-6-one:

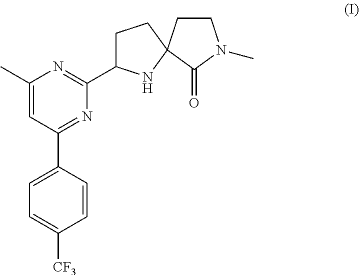

wherein said pharmaceutically acceptable salt thereof is selected from the citric acid (citrate) salt, methanesulfonic acid (mesylate) salt, sulfuric acid (hydrosulfate) salt, saccharin (saccharinate) salt and oxalic acid (oxalate) salt.

According to certain embodiments, the invention provides a compound of formula (I) which is a pharmaceutically acceptable salt of 7-methyl-2-[4-methyl-6-[4-(trifluoromethyl)-phenyl]pyrimidin-2-yl]-1,7-diazaspiro[4.4]nonan-6-one:

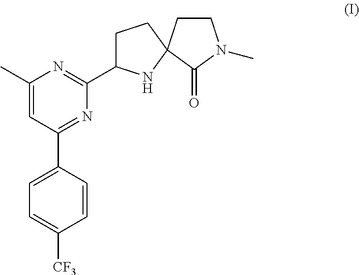

wherein said pharmaceutically acceptable salt thereof is selected from the citric acid (citrate) salt and methanesulfonic acid (mesylate) salt.

### BRIEF DESCRIPTION OF THE FIGURES

FIG. 1: XRPD patterns of citrate salt (Example 1), Ex-DVS examination at 0% RH, (top panel), 90% RH, (middle panel) and input (bottom panel).

FIG. 2: DSC and TGA thermographs of citrate salt (Example 1), heating rate 20° C.min⁻¹.

FIG. 3: XRPD data for mesylate salt (Example 2) exposed to extremes of humidity post GVS cycle: input (bottom panel) output 0% (top panel) output 90% (middle panel).

FIG. 4: DSC and TGA thermographs of mesylate salt (Example 2).

FIG. 5A: ORTEP representation of (2R,5S)-7-Methyl-2-(4-methyl-6-(4-(trifluoromethyl)phenyl)pyrimidin-2-yl)-1,7-diazaspiro[4.4]nonan-6-one hydrosulfate (Example 3).

FIG. 5B: XRPD data for (2R,5S)-7-Methyl-2-(4-methyl-6-(4-(trifluoromethyl)phenyl)pyrimidin-2-yl)-1,7-diazaspiro[4.4]nonan-6-one hydrosulfate salt (Example 3).

FIG. 6: XRPD data for (2R,5S)-7-Methyl-2-(4-methyl-6-(4-(trifluoromethyl)phenyl)pyrimidin-2-yl)-1,7-diazaspiro[4.4]nonan-6-one freebase (Example 4).

FIG. 7: XRPD data for (2R,5S)-7-Methyl-2-(4-methyl-6-(4-(trifluoromethyl)phenyl)pyrimidin-2-yl)-1,7-diazaspiro[4.4]nonan-6-one saccharinate (Example 5).

FIG. 8: XRPD data for (2R,5S)-7-Methyl-2-(4-methyl-6-(4-(trifluoromethyl)phenyl)pyrimidin-2-yl)-1,7-diazaspiro[4.4]nonan-6-one oxalate (Example 6).

### DETAILED DESCRIPTION OF THE INVENTION

A reference to a compound of the formula (I) and sub-groups thereof also includes ionic forms, solvates, isomers (including geometric and stereochemical isomers), tautomers, N-oxides, esters, prodrugs, isotopes and protected forms thereof, for example, as discussed below; preferably, the tautomers or isomers or N-oxides or solvates thereof; and more preferably, the tautomers or N-oxides or solvates thereof, even more preferably the tautomers or solvates thereof. Hereinafter, compounds and their ionic forms, solvates, isomers (including geometric and stereochemical isomers), tautomers, N-oxides, esters, prodrugs, isotopes and protected forms thereof as defined in any aspect of the invention (except intermediate compounds in chemical processes) are referred to as “compounds of the invention”.

The salts of the present invention can be synthesized from the parent base by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two; generally, nonaqueous media such as dichloromethane, 1,4-dioxane, ether, ethyl acetate, ethanol, isopropanol, or acetonitrile are used.

The compounds of the invention may exist as mono- or di-salts depending upon the pKa of the acid from which the salt is formed.

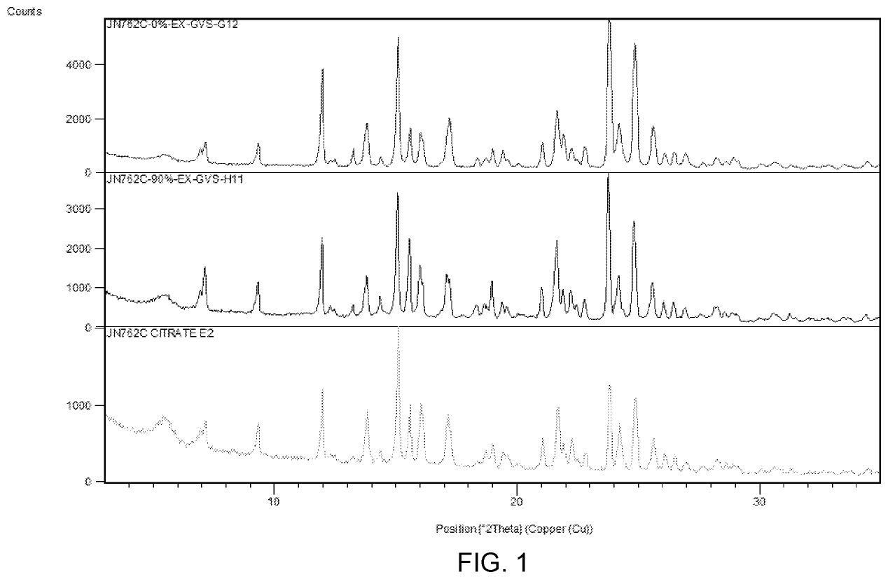

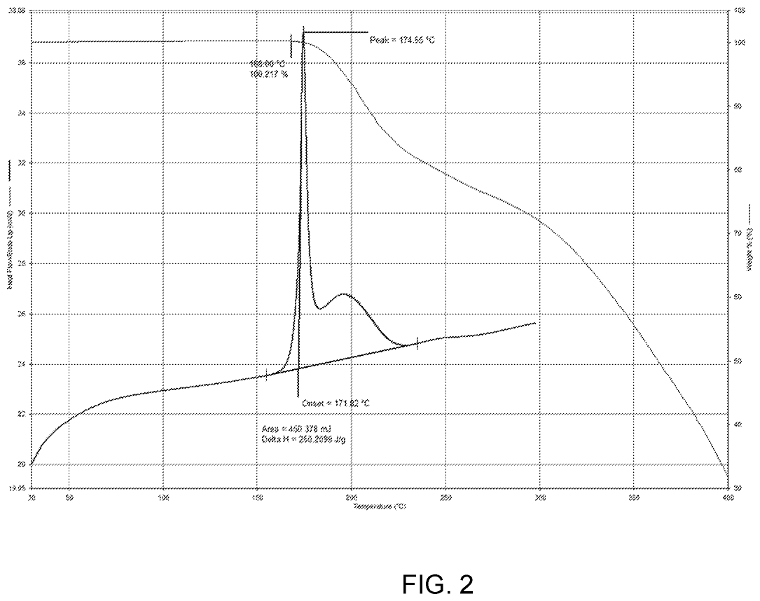

In one embodiment, the compound of formula (I) is (2R,5S)-7-Methyl-2-[4-methyl-6-[4-(trifluoromethyl)-phenyl]pyrimidin-2-yl]-1,7-diazaspiro[4.4]nonan-6-one citric acid (citrate) salt (E1). Data is presented herein in Example 1 and FIGS. 1 and 2 which demonstrate that under different extremes of humidity a stable crystalline form of the citrate salt of Example 1 shows no tendency to form hydrates (see XPRD data in FIG. 1). This is supported by DSC/TGA data in FIG. 2 which show clear transitions and no evidence of solvates. The compound of Example 1 also demonstrated a good level of aqueous solubility (22 mg/ml at 25° C.).

In one embodiment, the compound of formula (I) is (2R,5S)-7-Methyl-2-[4-methyl-6-[4-(trifluoromethyl)-phenyl]pyrimidin-2-yl]-1,7-diazaspiro[4.4]nonan-6-one citric acid (citrate) salt (E1) in a crystalline form. In a further embodiment, the crystalline form has 2θ values 15.2±0.2°, 23.7±0.2° and 24.8±0.2°. In a further embodiment, the crystalline form has 2θ values 12.0±0.2°, 15.2±0.2°, 15.7±0.2°, 21.7±0.2°, 23.7±0.2° and 24.8±0.2°. In a further embodiment, the crystalline form has an XRPD pattern substantially as shown in FIG. 1.

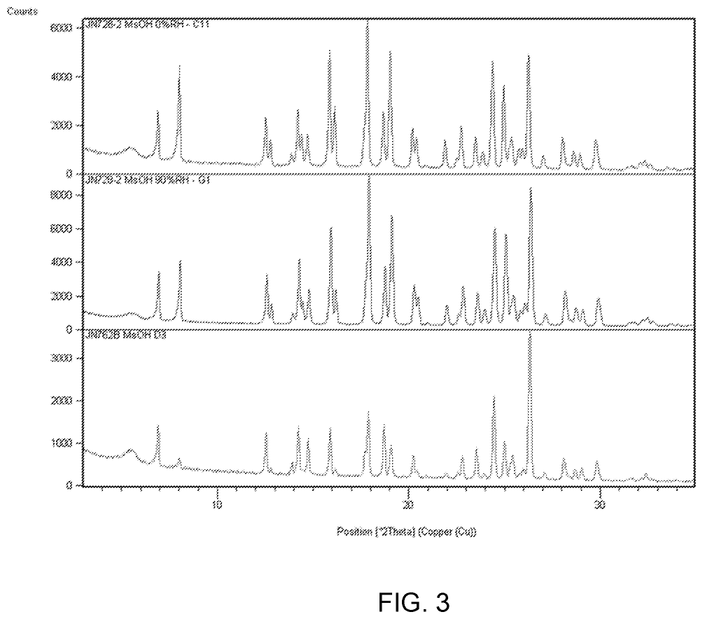

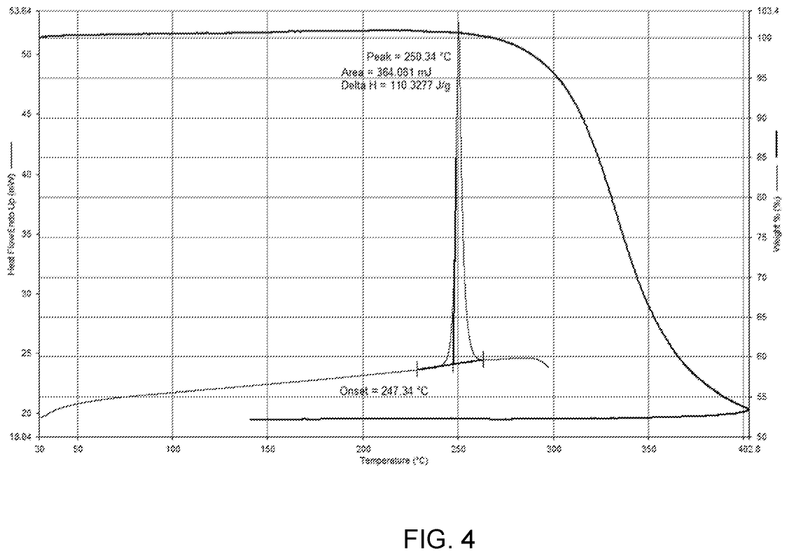

In an alternative embodiment, the compound of formula (I) is (2R,5S)-7-Methyl-2-[4-methyl-6-[4-(trifluoromethyl)-phenyl]pyrimidin-2-yl]-1,7-diazaspiro[4.4]nonan-6-one methanesulfonic acid (mesylate) salt (E2). Data is presented herein in Example 2 and FIGS. 3 and 4 which demonstrate that under different extremes of humidity a stable crystalline form of the mesylate salt of Example 2 shows no tendency to form hydrates (see XPRD data in FIG. 3). This is supported by DSC/TGA data in FIG. 4 which show clear transitions and no evidence of solvates. The compound of Example 2 also demonstrated a good level of aqueous solubility (65 mg/ml at 25° C.).

In one embodiment, the compound of formula (I) is (2R,5S)-7-Methyl-2-[4-methyl-6-[4-(trifluoromethyl)-phenyl]pyrimidin-2-yl]-1,7-diazaspiro[4.4]nonan-6-one methanesulfonic acid (mesylate) salt (E2) is in a crystalline form. In a further embodiment, the crystalline form has 2θ values 17.9±0.2°, 24.5±0.2° and 26.3±0.2°. In a further embodiment, the crystalline form has 2θ values 15.8±0.2°, 17.9±0.2°, 19.1±0.2°, 24.5±0.2°, 25.1±0.2° and 26.3±0.2°. In a further embodiment, the crystalline form has an XRPD pattern substantially as shown in FIG. 3.

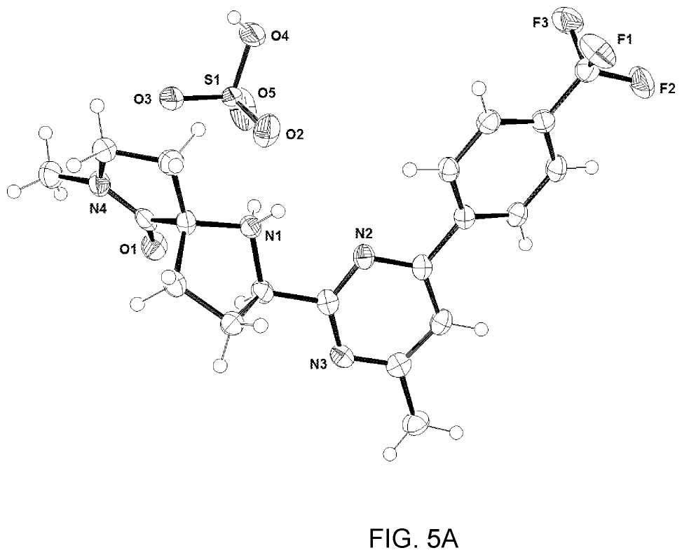

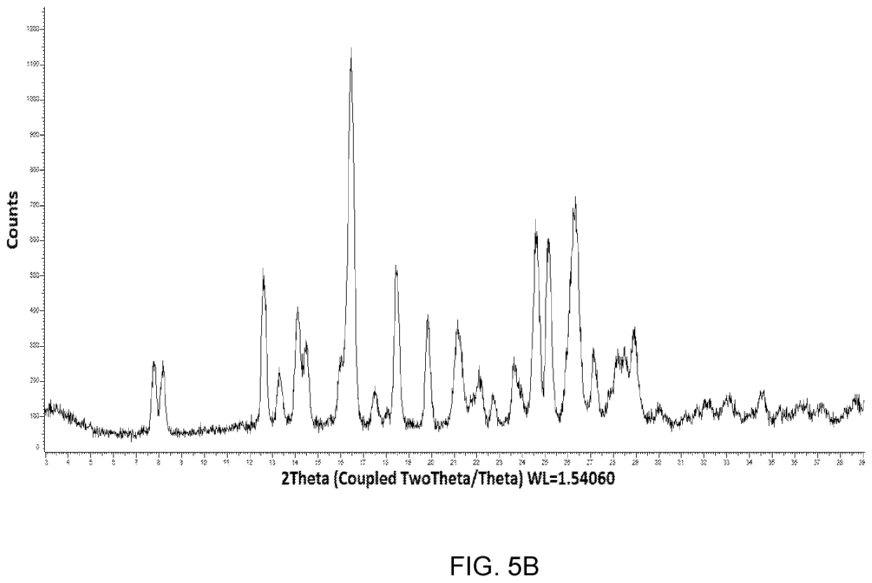

In an alternative embodiment, the compound of formula (I) is (2R,5S)-7-Methyl-2-[4-methyl-6-[4-(trifluoromethyl)-phenyl]pyrimidin-2-yl]-1,7-diazaspiro[4.4]nonan-6-one sulfuric acid (hydrosulfate) salt (E3), the preparation of which is demonstrated in Example 3 and FIGS. 5A and 5B.

In one embodiment, the compound of formula (I) is (2R,5S)-7-Methyl-2-[4-methyl-6-[4-(trifluoromethyl)-phenyl]pyrimidin-2-yl]-1,7-diazaspiro[4.4]nonan-6-one sulfuric acid (hydrosulfate) salt (E3) in a crystalline form. In a further embodiment, the crystalline form has four or more 2θ values selected from the group consisting of 8.1±0.2°, 12.6±0.2°, 14.3±0.2°, 16.5±0.2°, 18.5±0.2°, and 24.8±0.2°. In a further embodiment, the crystalline form has five or more 2θ values selected from the group consisting of 7.8±0.2°, 8.1±0.2°, 12.6±0.2°, 14.3±0.2°, 16.5±0.2°, 18.5±0.2°, 19.6±0.2°, 24.8±0.2° and 25.3±0.2°. In a further embodiment, the crystalline form has 2θ values 16.5±0.2°, 24.8±0.2°, and 25.3±0.2°. In a further embodiment, the crystalline form has 2θ values 12.6±0.2°, 16.5±0.2°, 18.5±0.2°, 24.8±0.2°, and 25.3±0.2°. In a further embodiment, the crystalline form has an XRPD pattern substantially as shown in FIG. 5B.

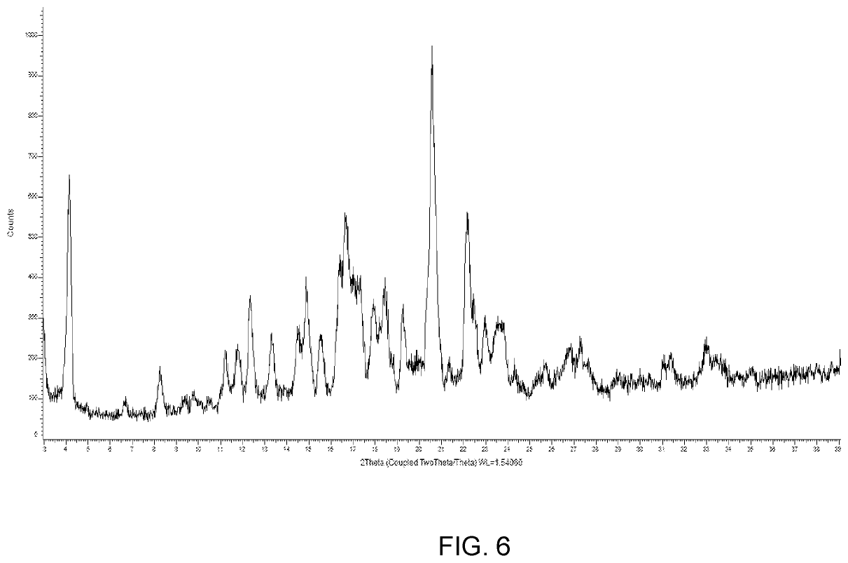

In an alternative embodiment, the compound of formula (I) is (2R,5S)-7-Methyl-2-[4-methyl-6-[4-(trifluoromethyl)-phenyl]pyrimidin-2-yl]-1,7-diazaspiro[4.4]nonan-6-one free base (E4), the preparation of which is demonstrated in Example 4 and FIG. 6.

In one embodiment, the compound of formula (I) is (2R,5S)-7-Methyl-2-[4-methyl-6-[4-(trifluoromethyl)-phenyl]pyrimidin-2-yl]-1,7-diazaspiro[4.4]nonan-6-one free base (E4) in a crystalline form. In a further embodiment, the crystalline form has 2θ values 4.1±0.2°, 17.0±0.2°, 20.8±0.2° and 22.5±0.2°. In a further embodiment, the crystalline form has 2θ values 4.1±0.2°, 12.5±0.2°, 14.9±0.2°, 17.0±0.2°, 20.8±0.2° and 22.5±0.2°. In a further embodiment, the crystalline form has an XRPD pattern substantially as shown in FIG. 6.

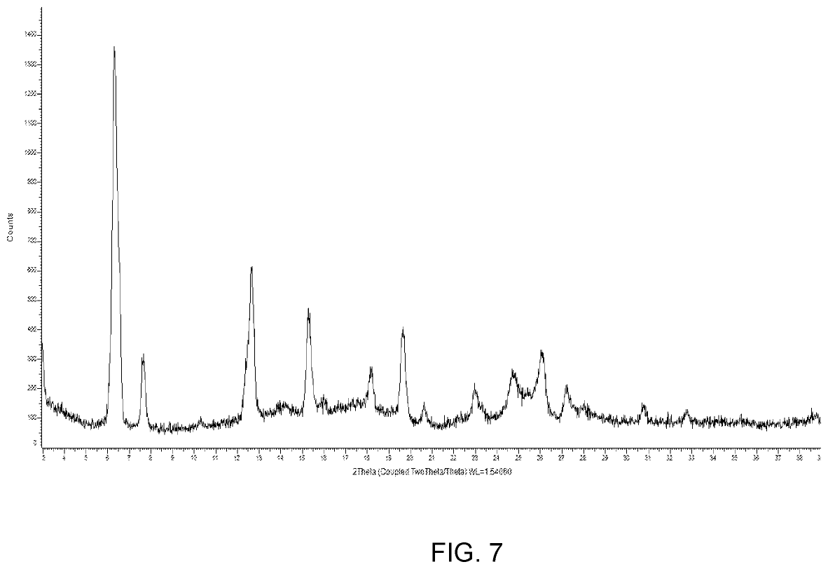

In an alternative embodiment, the compound of formula (I) is (2R,5S)-7-Methyl-2-[4-methyl-6-[4-(trifluoromethyl)-phenyl]pyrimidin-2-yl]-1,7-diazaspiro[4.4]nonan-6-one saccharin (saccharinate) salt (E5), the preparation of which is demonstrated in Example 5 and FIG. 7.

In one embodiment, the compound of formula (I) is (2R,5S)-7-Methyl-2-[4-methyl-6-[4-(trifluoromethyl)-phenyl]pyrimidin-2-yl]-1,7-diazaspiro[4.4]nonan-6-one saccharin (saccharinate) salt (E5) in a crystalline form. In a further embodiment, the crystalline form has 2θ values 6.4±0.2°, 12.8±0.2° and 15.4±0.2°. In a further embodiment, the crystalline form has 2θ values 6.4±0.2°, 7.7±0.2°, 12.8±0.2°, 15.4±0.2°, 19.8±0.2° and 26.3±0.2°. In a further embodiment, the crystalline form has an XRPD pattern substantially as shown in FIG. 7.

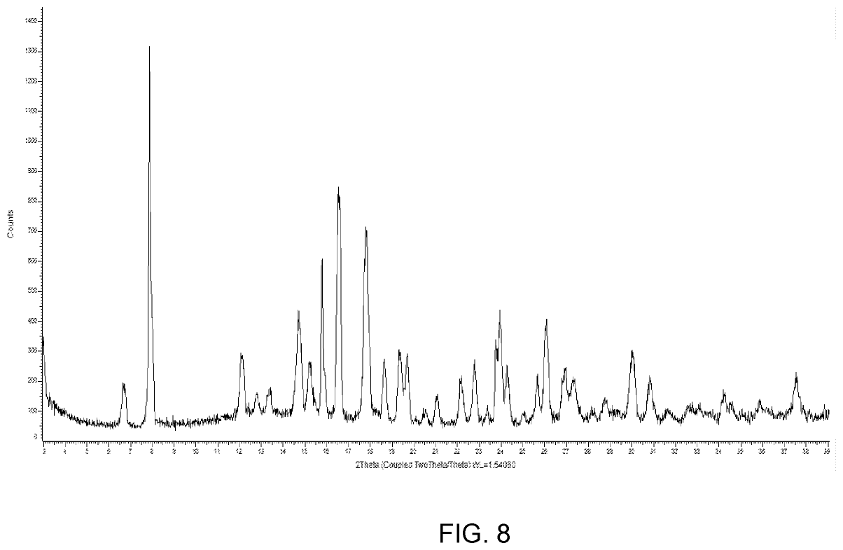

In an alternative embodiment, the compound of formula (I) is (2R,5S)-7-Methyl-2-[4-methyl-6-[4-(trifluoromethyl)-phenyl]pyrimidin-2-yl]-1,7-diazaspiro[4.4]nonan-6-one oxalic acid (oxalate) salt (E6), the preparation of which is demonstrated in Example 6 and FIG. 8.

In one embodiment, the compound of formula (I) is (2R,5S)-7-Methyl-2-[4-methyl-6-[4-(trifluoromethyl)-phenyl]pyrimidin-2-yl]-1,7-diazaspiro[4.4]nonan-6-one oxalic acid (oxalate) salt (E6) is in a crystalline form. In a further embodiment, the crystalline form has 2θ values 7.9±0.2°, 16.0±0.2° and 16.7±0.2°. In a further embodiment, the crystalline form has 2θ values 7.9±0.2°, 14.8±0.2°, 16.0±0.2°, 16.7±0.2°, 17.8±0.2°, 24.3±0.2° and 26.4±0.2°. In a further embodiment, the crystalline form has an XRPD pattern substantially as shown in FIG. 8.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Pharmaceutically acceptable solvates of the compound of the invention are within the scope of the invention.

Compounds of the formula (I) containing an amine function may also form N-oxides. A reference herein to a compound of the formula (I) that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

It will be appreciated by those skilled in the art that certain protected derivatives of compounds of formula (I), which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. All such prodrugs of compounds of the invention are included within the scope of the invention. Examples of pro-drug functionality suitable for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention.

Also included within the scope of the salts of the invention are polymorphs thereof. In certain embodiments, the polymorph of the crystalline compound is characterized by powder X-ray diffraction (XRD, XRPD, or pXRD). θ represents the diffraction angle, measured in degrees. In certain embodiments, the diffractometer used in XRD measures the diffraction angle as two times the diffraction angle θ. Thus, in certain embodiments, the diffraction patterns described herein refer to X-ray intensity measured against angle 2θ. Those of skill in the art will recognize that the exact location of the peaks in an XRPD pattern are subject to experimental uncertainty that is dependant upon the measurement conditions employed. It should be further understood that the relative intensities may also vary depending upon the experimental conditions and, accordingly, the exact order of intensity should not be taken into account.

Compounds of the formula (I) may exist in a number of different geometric isomeric, and tautomeric forms and references to compounds of the formula (I) include all such forms. For the avoidance of doubt, where a compound can exist in one of several geometric isomeric or tautomeric forms and only one is specifically described or shown, all others are nevertheless embraced by formula (I).

In one embodiment, the invention provides compounds of any one of formulae (Ia)-(Id):

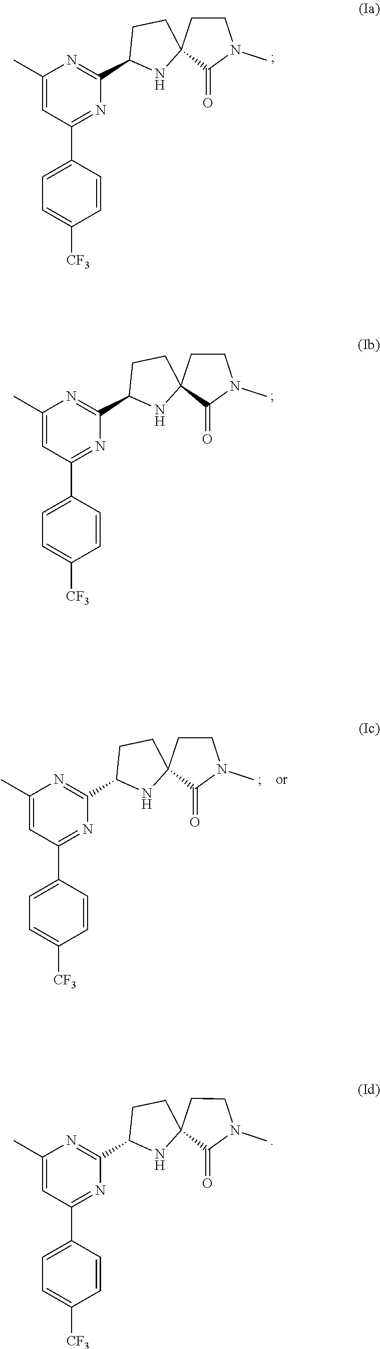

In a further embodiment, the invention provides compounds of formula (Ia). Representative examples of compounds of formula (Ia) include Examples 1-2 described herein.

The present invention includes all pharmaceutically acceptable isotopically-labeled compounds of the invention, i.e. compounds of formula (I), wherein one or more atoms are replaced by atoms having the same atomic number, but an atomic mass or mass number different from the atomic mass or mass number usually found in nature.

Examples of isotopes suitable for inclusion in the compounds of the invention comprise isotopes of hydrogen, such as ²H (D) and ³H (T), carbon, such as ¹¹C, ¹³C and ¹⁴C, fluorine, such as ¹⁸F, nitrogen, such as ¹³N and ¹⁵N, oxygen, such as ¹⁵O, ¹⁷O and ¹⁸O, and sulfur, such as ³⁵S.

Certain isotopically-labelled compounds of formula (I), for example, those incorporating a radioactive isotope, are useful in drug and/or substrate tissue distribution studies. The compounds of formula (I) can also have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase), etc. The radioactive isotopes tritium, i.e. ³H (T), and carbon-14, i.e. ¹⁴C, are particularly useful for this purpose in view of their ease of incorporation and ready means of detection.

Substitution with heavier isotopes such as deuterium, i.e. ²H (D), may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining target occupancy.

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

As discussed hereinabove, it is believed that compounds of the invention may be useful for the treatment of diseases and conditions mediated by modulation of voltage-gated sodium channels.

In one embodiment, the compounds will be state-dependent sodium channel inhibitors.

In another embodiment, the compounds will be subtype NaV1.7 sodium channel state-dependent inhibitors.

In another embodiment, the compounds will be state-dependent sodium channel inhibitors which have a suitable developability profile on oral administration, for example in terms of exposure (Cmax) and/or bioavailability.

In one embodiment, the compounds will be sodium channel inhibitors.

In another embodiment, the compounds will be subtype NaV1.7 sodium channel inhibitors.

In another embodiment, the compounds will be sodium channel inhibitors which have a suitable developability profile on oral administration, for example in terms of exposure (Cmax) and/or bioavailability.

According to a further aspect of the invention, there is provided compounds of the invention for use as a medicament, preferably a human medicament.

According to a further aspect the invention provides the use of compounds of the invention in the manufacture of a medicament for treating or preventing a disease or condition mediated by modulation of voltage-gated sodium channels.

In one particular embodiment, compounds of the invention may be useful as analgesics. For example they may be useful in the treatment of chronic inflammatory pain (e.g. pain associated with rheumatoid arthritis, osteoarthritis, rheumatoid spondylitis, gouty arthritis and juvenile arthritis); musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea.

Compounds of the invention may be useful in the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy; post-herpetic neuralgia; trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are incredibly heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Compounds of the invention may also be useful in the amelioration of inflammatory disorders, for example in the treatment of skin conditions (e.g. sunburn, burns, eczema, dermatitis, psoriasis); ophthalmic diseases; lung disorders (e.g. asthma, bronchitis, emphysema, allergic rhinitis, non-allergic rhinitis, cough, respiratory distress syndrome, pigeon fancier's disease, farmer's lung, chronic obstructive pulmonary disease, (COPD); gastrointestinal tract disorders (e.g. Crohn's disease, ulcerative colitis, coeliac disease, regional ileitis, irritable bowel syndrome, inflammatory bowel disease, gastroesophageal reflux disease); other conditions with an inflammatory component such as migraine, multiple sclerosis, myocardial ischemia.

In one embodiment, the compounds of the invention are useful in the treatment of neuropathic pain or inflammatory pain as described herein.

Without wishing to be bound by theory, other diseases or conditions that may be mediated by modulation of voltage-gated sodium channels are selected from the list consisting of [the numbers in brackets after the listed diseases below refer to the classification code in Diagnostic and Statistical Manual of Mental Disorders, 4th Edition, published by the American Psychiatric Association (DSM-IV) and/or the International Classification of Diseases, 10th Edition (ICD-10)]:

i) Depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90):

ii) Schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).

iii) Anxiety disorders including Panic Attack; Panic Disorder including Panic Disorder without Agoraphobia (300.01) and Panic Disorder with Agoraphobia (300.21); Agoraphobia; Agoraphobia Without History of Panic Disorder (300.22), Specific Phobia (300.29, formerly Simple Phobia) including the subtypes Animal Type, Natural Environment Type, Blood-Injection-Injury Type, Situational Type and Other Type), Social Phobia (Social Anxiety Disorder, 300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder, Separation Anxiety Disorder (309.21), Adjustment Disorders with Anxiety (309.24) and Anxiety Disorder Not Otherwise Specified (300.00):

iv) Substance-related disorders including Substance Use Disorders such as Substance Dependence, Substance Craving and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide:

v) Enhancement of cognition including the treatment of cognition impairment in other diseases such as schizophrenia, bipolar disorder, depression, other psychiatric disorders and psychotic conditions associated with cognitive impairment, e.g. Alzheimer's disease:

vi) Sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; sleep apnea and jet-lag syndrome:

vi) Eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; Binge Eating Disorder; and Eating Disorder Not Otherwise Specified (307.50):

vii) Autism Spectrum Disorders including Autistic Disorder (299.00), Asperger's Disorder (299.80), Rett's Disorder (299.80), Childhood Disintegrative Disorder (299.10) and Pervasive Disorder Not Otherwise Specified (299.80, including Atypical Autism).

viii) Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit /Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23):

ix) Personality Disorders including the subtypes Paranoid Personality Disorder (301.0), Schizoid Personality Disorder (301.20), Schizotypal Personality Disorder (301,22), Antisocial Personality Disorder (301.7), Borderline Personality Disorder (301,83), Histrionic Personality Disorder (301.50), Narcissistic Personality Disorder (301,81), Avoidant Personality Disorder (301.82), Dependent Personality Disorder (301.6), Obsessive-Compulsive Personality Disorder (301.4) and Personality Disorder Not Otherwise Specified (301.9): and

x) Sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

xi) Impulse control disorder” including: Intermittent Explosive Disorder (312.34), Kleptomania (312.32), Pathological Gambling (312.31), Pyromania (312.33), Trichotillomania (312.39), Impulse-Control Disorders Not Otherwise Specified (312.3), Binge Eating, Compulsive Buying, Compulsive Sexual Behaviour and Compulsive Hoarding.

In another embodiment, diseases or conditions that may be mediated by modulation of voltage gated sodium channels are depression or mood disorders

In another embodiment, diseases or conditions that may be mediated by modulation of voltage gated sodium channels are substance related disorders.

In a further embodiment, diseases or conditions that may be mediated by modulation of voltage gated sodium channels are Bipolar Disorders (including Bipolar I Disorder, Bipolar II Disorder (i.e. Recurrent Major Depressive Episodes with Hypomanic Episodes) (296.89), Cyclothymic Disorder (301.13) or Bipolar Disorder Not Otherwise Specified (296.80)).

In a still further embodiment, diseases or conditions that may be mediated by modulation of voltage gated sodium channels are Nicotine-Related Disorders such as Nicotine Dependence (305.1), Nicotine Withdrawal (292.0) or Nicotine-Related Disorder Not Otherwise Specified (292.9).

Compounds of the invention may also be useful in the treatment and/or prevention of disorders treatable and/or preventable with anti-convulsive agents, such as epilepsy including post-traumatic epilepsy, obsessive compulsive disorders (OCD), sleep disorders (including circadian rhythm disorders, insomnia & narcolepsy), tics (e.g. Giles de la Tourette's syndrome), ataxias, muscular rigidity (spasticity), and temporomandibular joint dysfunction.

Compounds of the invention may also be useful in the treatment of bladder hyperrelexia following bladder inflammation.

Compounds of the invention may also be useful in the treatment of neurodegenerative diseases and neurodegeneration such as dementia, particularly degenerative dementia (including senile dementia, Alzheimer's disease, Pick's disease, Huntington's chorea, Parkinson's disease and Creutzfeldt-Jakob disease, motor neuron disease); The compounds may also be useful for the treatment of amyotrophic lateral sclerosis (ALS) and neuroinflamation.

Compounds of the invention may also be useful in neuroprotection and in the treatment of neurodegeneration following stroke, cardiac arrest, pulmonary bypass, traumatic brain injury, spinal cord injury or the like.

Compounds of the invention may also be useful in the treatment of tinnitus, and as local anaesthetics.

The compounds of the invention may also be used in combination with other therapeutic agents. The invention thus provides, in a further aspect, a combination comprising a compound of the invention or a pharmaceutically acceptable derivative thereof together with a further therapeutic agent.

When a compound of the invention or a pharmaceutically acceptable derivative thereof is used in combination with a second therapeutic agent active against the same disease state the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations by any convenient route.

When administration is sequential, either the compound of the invention or the second therapeutic agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art.

When used in the treatment or prophylaxis of pain, the compound of formula (I) or a pharmaceutically acceptable salt thereof may be used in combination with other medicaments indicated to be useful in the treatment or prophylaxis of pain of neuropathic origin including neuralgias, neuritis and back pain, and inflammatory pain including osteoarthritis, rheumatoid arthritis, acute inflammatory pain, back pain and migraine. Such therapeutic agents include for example COX-2 (cyclooxygenase-2) inhibitors, such as celecoxib, deracoxib, rofecoxib, valdecoxib, parecoxib, COX-189 or 2-(4-ethoxy-phenyl)-3-(4-methanesulfonyl-phenyl)-pyrazolo[1,5-b]pyridazine (WO 99/012930); 5-lipoxygenase inhibitors; NSAIDs (non-steroidal anti-inflammatory drugs) such as diclofenac, indomethacin, nabumetone or ibuprofen; bisphosphonates, leukotriene receptor antagonists; DMARDs (disease modifying anti-rheumatic drugs) such as methotrexate; adenosine A1 receptor agonists; sodium channel blockers, such as lamotrigine; NMDA (N-methyl-D-aspartate) receptor modulators, such as glycine receptor antagonists or memantine; ligands for the α₂δ-subunit of voltage gated calcium channels, such as gabapentin, pregabalin and solzira; tricyclic antidepressants such as amitriptyline; neurone stabilising antiepileptic drugs; cholinesterase inhibitors such as galantamine; mono-aminergic uptake inhibitors such as venlafaxine; opioid analgesics; local anaesthetics; 5HT₁ agonists, such as triptans, for example sumatriptan, naratriptan, zolmitriptan, eletriptan, frovatriptan, almotriptan or rizatriptan; nicotinic acetyl choline (nACh) receptor modulators; glutamate receptor modulators, for example modulators of the NR2B subtype; EP₄ receptor ligands; EP₂ receptor ligands; EP₃ receptor ligands; EP₄ agonists and EP₂ agonists; EP₄ antagonists; EP₂ antagonists and EP₃ antagonists; cannabinoid receptor ligands; bradykinin receptor ligands; vanilloid receptor or Transient Receptor Potential (TRP) ligands; and purinergic receptor ligands, including antagonists at P2X₃, P2X_{2/3}, P2X₄, P2X₇ or P2X_{4/7}; KCNQ/Kv7 channel openers, such as retigabine; additional COX-2 inhibitors are disclosed in U.S. Pat. Nos. 5,474,995, 5,633,272, 5,466,823, 6,310,099 and 6,291,523; and in WO 96/25405, WO 97/38986, WO 98/03484, WO 97/14691, WO 99/12930, WO 00/26216, WO 00/52008, WO 00/38311, WO 01/58881 and WO 02/18374.

The compounds of the invention may be used in combination with the following agents to treat or prevent psychotic disorders: i) antipsychotics; ii) drugs for extrapyramidal side effects, for example anticholinergics (such as benztropine, biperiden, procyclidine and trihexyphenidyl), antihistamines (such as diphenhydramine) and dopaminergics (such as amantadine); iii) antidepressants; iv) anxiolytics; and v) cognitive enhancers for example cholinesterase inhibitors (such as tacrine, donepezil, rivastigmine and galantamine).

The compounds of the invention may be used in combination with the following agents to treat or prevent psychotic disorders: i) antipsychotics; ii) drugs for extrapyramidal side effects, for example anticholinergics (such as benztropine, biperiden, procyclidine and trihexyphenidyl), antihistamines (such as diphenhydramine) and dopaminergics (such as amantadine); iii) antidepressants; iv) anxiolytics; and v) cognitive enhancers for example cholinesterase inhibitors (such as tacrine, donepezil, rivastigmine and galantamine).

The compounds of the invention may be used in combination with antidepressants to treat or prevent depression and mood disorders.

The compounds of the invention may be used in combination with the following agents to treat or prevent bipolar disease: i) mood stabilisers; ii) antipsychotics; and iii) antidepressants.

The compounds of the invention may be used in combination with the following agents to treat or prevent anxiety disorders: i) anxiolytics; and ii) antidepressants.

The compounds of the invention may be used in combination with the following agents to improve nicotine withdrawal and reduce nicotine craving: i) nicotine replacement therapy for example a sublingual formulation of nicotine beta-cyclodextrin and nicotine patches; and ii) bupropion.

The compounds of the invention may be used in combination with the following agents to improve alcohol withdrawal and reduce alcohol craving: i) NMDA receptor antagonists for example acamprosate; ii) GABA receptor agonists for example tetrabamate; and iii) Opioid receptor antagonists for example naltrexone.

The compounds of the invention may be used in combination with the following agents to improve opiate withdrawal and reduce opiate craving: i) opioid mu receptor agonist/opioid kappa receptor antagonist for example buprenorphine; ii) opioid receptor antagonists for example naltrexone; and iii) vasodilatory antihypertensives for example lofexidine.

The compounds of the invention may be used in combination with the following agents to treat or prevent sleeping disorders: i) benzodiazepines for example temazepam, lormetazepam, estazolam and triazolam; ii) non-benzodiazepine hypnotics for example zolpidem, zopiclone, zaleplon and indiplon; iii) barbiturates for example aprobarbital, butabarbital, pentobarbital, secobarbita and phenobarbital; iv) antidepressants; v) other sedative-hypnotics for example chloral hydrate and chlormethiazole.

The compounds of the invention may be used in combination with the following agents to treat anorexia: i) appetite stimulants for example cyproheptidine; ii) antidepressants; iii) antipsychotics; iv) zinc; and v) premenstral agents for example pyridoxine and progesterones.

The compounds of the invention may be used in combination with the following agents to treat or prevent bulimia: i) antidepressants; ii) opioid receptor antagonists; iii) antiemetics for example ondansetron; iv) testosterone receptor antagonists for example flutamide; v) mood stabilisers; vi) zinc; and vii) premenstral agents.

The compounds of the invention may be used in combination with the following agents to treat or prevent autism: i) antipsychotics; ii) antidepressants; iii) anxiolytics; and iv) stimulants for example methylphenidate, amphetamine formulations and pemoline.

The compounds of the invention may be used in combination with the following agents to treat or prevent ADHD: i) stimulants for example methylphenidate, amphetamine formulations and pemoline; and ii) non-stimulants for example norepinephrine reuptake inhibitors (such as atomoxetine), alpha 2 adrenoceptor agonists (such as clonidine), antidepressants, modafinil, and cholinesterase inhibitors (such as galantamine and donezepil).

The compounds of the invention may be used in combination with the following agents to treat personality disorders: i) antipsychotics; ii) antidepressants; iii) mood stabilisers; and iv) anxiolytics.

The compounds of the invention may be used in combination with the following agents to treat or prevent male sexual dysfunction: i) phosphodiesterase V inhibitors, for example vardenafil and sildenafil; ii) dopamine agonists/dopamine transport inhibitors for example apomorphine and buproprion; iii) alpha adrenoceptor antagonists for example phentolamine; iv) prostaglandin agonists for example alprostadil; v) testosterone agonists such as testosterone; vi) serotonin transport inhibitors for example serotonin reuptake inhibitors; v) noradrenaline transport inhibitors for example reboxetine and vii) 5-HT1A agonists, for example flibanserine.

The compounds of the invention may be used in combination with the same agents specified for male sexual dysfunction to treat or prevent female sexual dysfunction, and in addition an estrogen agonist such as estradiol.

Antipsychotic drugs include: Typical Antipsychotics (for example chlorpromazine, thioridazine, mesoridazine, fluphenazine, perphenazine, prochlorperazine, trifluoperazine, thiothixine, haloperidol, molindone and loxapine); and Atypical Antipsychotics (for example clozapine, olanzapine, risperidone, quetiapine, aripirazole, ziprasidone and amisulpride).

Antidepressant drugs include serotonin reuptake inhibitors (such as citalopram, escitalopram, fluoxetine, paroxetine and sertraline); dual serotonin/noradrenaline reuptake inhibitors (such as venlafaxine, duloxetine and milnacipran); Noradrenaline reuptake inhibitors (such as reboxetine); tricyclic antidepressants (such as amitriptyline, clomipramine, imipramine, maprotiline, nortriptyline and trimipramine); monoamine oxidase inhibitors (such as isocarboxazide, moclobemide, phenelzine and tranylcypromine); and others (such as bupropion, mianserin, mirtazapine, nefazodone and trazodone).

Mood stabiliser drugs include lithium, sodium valproate/valproic acid/divalproex, carbamazepine, lamotrigine, gabapentin, topiramate and tiagabine.

Anxiolytics include benzodiazepines such as alprazolam and lorazepam.

It will be appreciated that references herein to “treatment” extend to prophylaxis, prevention of recurrence and suppression or amelioration of symptoms (whether mild, moderate or severe) as well as the treatment of established conditions.

The compound of the invention may be administered as the raw chemical but the active ingredient is preferably presented as a pharmaceutical formulation.

According to a further aspect, the invention provides a pharmaceutical composition comprising a compound of the invention, in association with one or more pharmaceutically acceptable carrier(s), diluents(s) and/or excipient(s). The carrier, diluent and/or excipient must be “acceptable” in the sense of being compatible with the other ingredients of the composition and not deleterious to the recipient thereof.

The compounds of the invention may be administered in conventional dosage forms prepared by combining a compound of the invention with standard pharmaceutical carriers or diluents according to conventional procedures well known in the art. These procedures may involve mixing, granulating and compressing or dissolving the ingredients as appropriate to the desired preparation.

The pharmaceutical compositions of the invention may be formulated for administration by any route, and include those in a form adapted for oral, topical or parenteral administration to mammals including humans.

The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatine, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatine, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilising the compound and a sterile vehicle, water being preferred. The compound, depending on the vehicle and concentration used, can be either suspended or dissolved in the vehicle. In preparing solutions the compound can be dissolved in water for injection and filter-sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilised powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilisation cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, for example from 10-60% by weight, of the active material, depending on the method of administration. Where the compositions comprise dosage units, each unit will for example contain from 5-1000 mg of the active ingredient. The dosage as employed for adult human treatment may range from 10 to 3000 mg per day depending on the route and frequency of administration. For oral administration a typical dose may be in the range of 50 to 1500 mg per day, for example 120 to 1000 mg per day.

It will be recognised by one of skill in the art that the optimal quantity and spacing of individual dosages of a compound of the invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of a compound of the invention given per day for a defined number of days, can be ascertained by those skilled in the art using conventional course of treatment determination tests.

All publications, including, but not limited to, patents and patent applications cited in this specification, are herein incorporated by reference as if each individual publication were specifically and individually indicated to be incorporated by reference herein as though fully set forth.

It will be appreciated that the invention includes the following further aspects. The embodiments described for the first aspect similarly apply to these further aspects. The diseases and conditions described above extend, where appropriate, to these further aspects:
i) A compound of the invention for use in treating or preventing a disease or condition mediated by modulation of voltage-gated sodium channels.
ii) A method of treatment or prevention of a disease or condition mediated by modulation of voltage-gated sodium channels in a mammal comprising administering an effective amount of a compound of the invention.
iii) Use of a compound of the invention in the manufacture of a medicament to treat or prevent a disease or condition mediated by modulation of voltage-gated sodium channels.
iv) Use of a compound of the invention to treat or prevent a disease or condition mediated by modulation of voltage-gated sodium channels.

Experimental

General

Solution Proton NMR

¹H NMR spectra were collected using a JEOL ECX 400 MHz spectrometer equipped with an auto-sampler. The samples were dissolved in a suitable deuterated solvent for analysis. The data was acquired using Delta NMR Processing and Control Software version 4.3.

X-Ray Powder Diffraction (XRPD)

X-Ray Powder Diffraction patterns were collected on a PANalytical diffractometer using Cu Kα radiation (45 kV, 40 mA), θ−θ goniometer, focusing mirror, divergence slit (½″), soller slits at both incident and divergent beam (4 mm) and a PIXcel detector. The software used for data collection was X'Pert Data Collector, version 2.2f and the data was presented using X'Pert Data Viewer, version 1.2d.

XRPD patterns were acquired under ambient conditions via a transmission foil sample stage (polyimide—Kapton, 12.7 μm thickness film) under ambient conditions using a PANalytical X'Pert PRO. The data collection range was 2.994-35°2θ with a continuous scan speed of 0.202004°s⁻¹.

Single Crystal X-Ray Diffraction Parameters:

Single crystal analyses were performed using a Bruker APEX-II CCD diffractometer (173K)

Samples were mounted on a nylon loop with paratone oil for data collection using a MoKα radiation source. Using Olex2 (Dolomanov et al., 2009), the structure was solved with the SheIXS (Sheldrick, 2008) structure solution program, using the Direct Methods solution method. The model was refined with version 2014/6 of XL (Sheldrick, 2008) using Least Squares minimization.

Differential Scanning calorimetry (DSC)

DSC data was collected on a PerkinElmer Pyris 6000 DSC equipped with a 45 position sample holder. The instrument was verified for energy and temperature calibration using certified indium. A predefined amount of the sample, 0.5-3.0 mg, was placed in a pin holed aluminium pan and heated at 20° C.min⁻¹ from 30 to 300° C., or a higher temperature if required. A purge of dry nitrogen at 20 ml.min⁻¹ was maintained over the sample. The instrument control, data acquisition and analysis was performed with Pyris Software v11.1.1 revision H.

Thermo-Gravimetric Analysis (TGA)

TGA data were collected on a PerkinElmer Pyris 1 TGA equipped with a 20 position auto-sampler. The instrument was calibrated using a certified weight and certified Alumel and Perkalloy for temperature. A predefined amount of the sample, 1-5 mg, was loaded onto a pre-tared aluminium crucible and was heated at 20° C.min⁻¹ from ambient temperature to 400° C. A nitrogen purge at 20 ml.min⁻¹ was maintained over the sample. The instrument control, data acquisition and analysis was performed with Pyris Software v11.1.1 revision H.

Solubility

Samples of each salt were equilibrated (magnetic agitation) at a constant temperature of 20° C. in a volume of 1.2 ml de-ionised water for a total of 24 hours. Solids were isolated by filtration to deliver clear liquors that were analyzed for API content by HPLC vs a known standard of free base (typical dilution of 50× using deionized water for salt candidates to deliver a working concentration in line with the standard at 0.5 mg/ml). The solids were dried at 50° C. and re-analysed by XRPD to confirm form retention—both mesylate and citrate retain form.

### EXAMPLES

The invention is illustrated by the Examples described below.

### Example 1

### (2R,5S)-7-Methyl-2-[4-methyl-6-[4-(trifluoromethyl)-phenyl]pyrimidin-2-yl]-1,7-diazaspiro[4.4]nonan-6-one citric acid (citrate) salt (E1)

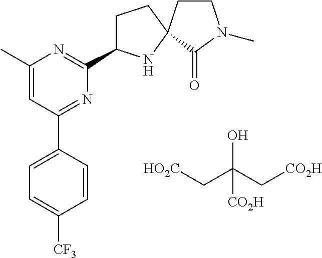

To a solution of (2R,5S)-7-methyl-2-[4-methyl-6-[4-(trifluoromethyl)-phenyl]pyrimidin-2-yl]-1,7-diazaspiro[4.4]nonan-6-one (which may be prepared in accordance with the procedure described in Example 1 of WO 2013/175205) (4.45 g, 0.0114 mol) dissolved in absolute ethanol (66.82 ml, 15 vol) at 45° C. was added a solution of citric acid in ethanol (1 M, 1.05 equiv. 12 ml) over a period of 2-3 minutes. The solution was aged at 45° C. for a period of 1 hour. After 30 minutes a seed of citrate salt (0.1 wt %) was added and the mixture allowed to cool over approximately 2 hours and mature for 18 hours at ambient temperature (approximately 10-15° C.). Following maturation the salt was noted to be a very thick suspension (white) that required mobilisation with 20 ml additional ethanol and a further maturation period of 2 hours at ambient temperature. Filtration was carried out under vacuum and the vessel and cake rinsed with 15 ml ethanol. The de-liquored cake was dried further in a vacuum oven at 50° C. to provide 6.0 g of crystalline white solid (91% yield).

¹H NMR (400 MHz, DMSO-D6): δ_{H} 1.90-2.05 (2H, m), 2.10-2.20 (2H, m,), 2.20-2.30 (1H, m), ˜2.50 (1H, m, partially masked by solvent)), 2.55-2.68 (4H, m), 2.56 (3H, s), 2.79 (3H, s), 3.28-3.40 (2H, m), 4.79 (1H, t, J=8.0 Hz), 7.92 (2H, d, J=8.4 Hz), 8.03 (1H, s), 8.45 (2H, d, J=8.8 Hz) ppm, (exchangeables not reported)

### Characterisation of Example 1

The XRPD of Example 1 is presented in FIG. 1 and the DSC/TGA of Example 1 is presented in FIG. 2. The citrate salt of Example 1 displayed the following characteristics:

| | |
|---|---|
| 1 endotherm | onset: 171.82° C. |
| | peak maximum: 174.55° C. |

There was an endotherm post the main endotherm.

There was no weight reduction until ca 168° C. had been reached. The weight reduction commenced with the start of the main endotherm and coincided with the endotherm post the main endotherm which indicated that this thermal event was the onset of compound decomposition and loss of citric acid. Thermal events >220° C. were due to compound decomposition.

The XPRD data in FIG. 1 demonstrated that under different extremes of humidity indicate a stable crystalline form of the citrate salt of Example 1 with no tendency to form hydrates. This is supported by DSC/TGA data in FIG. 2 which show clear transitions and no evidence of solvates.

Aqueous solubility of the citrate salt (Example 1)=22 mg/ml (25° C.).

### Example 2

### (2R,5S)-7-Methyl-2-[4-methyl-6-[4-(trifluoromethyl)-phenyl]pyrimidin-2-yl]-1,7-diazaspiro[4.4]nonan-6-one methanesulfonic acid (mesylate) salt (E2)

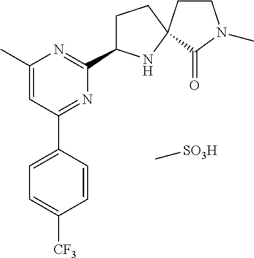

To a solution of (2R,5S)-7-methyl-2-[4-methyl-6-[4-(trifluoromethyl)-phenyl]pyrimidin-2-yl]-1,7-diazaspiro[4.4]nonan-6-one (which may be prepared in accordance with the procedure described in Example 1 of WO 2013/175205) (4.45 g, 0.0114 mol) dissolved in absolute ethanol (66.82 ml, 15 vol) at 45° C. was added a solution of methanesulfonic acid in ethanol (1 M, 1.05 equiv. 12 ml) over a period of 2-3 minutes. The solution was aged at 45° C. for a period of 1 hour. After 10 minutes nucleation and gradual crystallisation was noted to afford a thick mixture. Additional ethanol was added (10 ml) to mobilise the suspension that was then allowed to cool over approximately 2 hours and mature for 18 hours at ambient temperature (approximately 10-15° C.). Following maturation the salt was noted to be a thin, mobile suspension (white) that was filtered under vacuum and the vessel and cake rinsed with 15 ml ethanol. The de-liquored cake was dried further in a vacuum oven at 50° C. to provide 4.0 g of crystalline white solid (72% yield).

¹H NMR (400 MHz, DMSO-D6): δ_{H} 2.1-2.45 (4H, m), 2.27 (3H, s), 2.50-2.75 (2H, m), 2.61 (3H, s), 2.86 (3H, s), 3.35-3.50 (2H, m), 5.20 (1H, t, J=8 Hz), 7.96 (2H, d, J=8.8 Hz), 8.17 (1H, s), 8.51 (2H, d, J=8.4Hz), 9.45 (1H, br), 10.16 (1H, br) ppm.

### Characterisation of Example 2

The XRPD of Example 2 is presented in FIG. 3 and the DSC/TGA of Example 2 is presented in FIG. 4. The DSC thermograph of the methanesulfonate (mesylate) (Example 2) displayed the following characteristics:

| | |
|---|---|
| One distinct endotherm | onset: 247.34° C. |
| | peak maximum: 250.34° C. |

The TGA thermograph showed no weight reduction until ca 250° C. had been reached. The weight reduction commenced with the start of the main endotherm and indicated that this thermal event was the onset of compound decomposition. There is no evidence of entrapped solvents or water.

The XPRD data in FIG. 3 demonstrated that under different extremes of humidity indicate a stable crystalline form of the mesylate salt of Example 2 with no tendency to form hydrates. This is supported by DSC/TGA data in FIG. 4 which show clear transitions and no evidence of solvates.

Aqueous solubility of the mesylate salt (Example 2)=65 mg/ml (25° C.).

### Example 3

Preparation of (2R,5S)-7-methyl-2-(4-methyl-6-(4-(trifluoromethyl)phenyl)pyrimidin-2-yl)-1,7-diazaspiro[4.4]nonan-6-one hydrosulfate single crystals: 25.0 mg of (2R,5S)-7-Methyl-2-(4-methyl-6-(4-(trifluoromethyl)phenyl)pyrimidin-2-yl)-1,7-diazaspiro[4.4]nonan-6-one hydrosulfate was added to 4 mL vial. 1.000 mL of anhydrous EtOH was added, and the sample was filtered. Anhydrous hexanes were added dropwise until the solution neared the precipitation point. The vial was sealed and left undisturbed for 24 hr, after which time a crop of single crystals was evident. The sample was sent for single crystal analysis and confirmed as the anhydrous (2R,5S)-7-Methyl-2-(4-methyl-6-(4-(trifluoromethyl)phenyl)pyrimidin-2-yl)-1,7-diazaspiro[4.4]nonan-6-one hydrosulfate form (FIGS. 5A-5B).

### Example 4

Preparation of (2R,5S)-7-methyl-2-(4-methyl-6-(4-(trifluoromethyl)phenyl)pyrimidin-2-yl)-1,7-diazaspiro[4.4]nonan-6-one freebase: 8.00 g of (2R,5S)-7-Methyl-2-(4-methyl-6-(4-(trifluoromethyl)phenyl)pyrimidin-2-yl)-1,7-diazaspiro[4.4]nonan-6-one hydrosulfate (JM Lot R-2017-4323 D 301) was added to a 1 L Erlenmeyer flask and suspended and stirred vigorously in 400 mL of THF. 20% K2CO3 (250 mL) was added and dissolved. The mixture was transferred to 1 L sep. funnel. 100 mL EtOAc was added and the aqueous and organic layers were separated. The aqueous layer was re-extracted with 50 mL of EtOAc and the combined organics were back-extracted with brine (100 mL) and water (100 mL). Due to fairly poor separation, a significant quantity of MgSO₄ was required to dry the solution. The solution was reduced via Rotavap (45° C.) to ˜50 mL, transferred to a 100 mL RB flask, reduced down to ˜10 mL, transferred to 20 mL scintillation vial and continued to be reduced to a thick oil. The oil was left on the Rotavap for another hour and a “wet” solid was obtained. Loosened solids on the bottom of the vial were left on the Rotavap for 1 hr with no heat applied to obtain a chunky solid. The contents was transferred to a mortar and pestle, ground to powder and fine granules, placed back in a 20 mL scintillation vial and left on a Rotavap overnight to obtain a dry solid (5.1 g). The XRPD pattern of (2R,5S)-7-Methyl-2-(4-methyl-6-(4-(trifluoromethyl)phenyl)pyrimidin-2-yl)-1,7-diazaspiro[4.4]nonan-6-one freebase is shown in FIG. 6.

### Example 5

Preparation of (2R,5S)-7-methyl-2-(4-methyl-6-(4-(trifluoromethyl)phenyl)pyrimidin-2-yl)-1,7-diazaspiro[4.4]nonan-6-one saccharinate: 199.7 mg of (2R,5S)-7-Methyl-2-(4-methyl-6-(4-(trifluoromethyl)phenyl)pyrimidin-2-yl)-1,7-diazaspiro[4.4]nonan-6-one free base (0.5115 mmol) was dissolved in 4.2 mL of 2-Me-THF. 98.1 mg of saccharin (0.5106 mmol) was dissolved in 4.2 mL of 2-Me-THF. Saccharin was added to the freebase, and after 15 seconds the mixture began to precipitate and solidify. 10 mL of 2-Me-THF was added and stirred at max rpm as to provide a thick white suspension in 10 min. The suspension was filtered, air dried under vacuum for 10 min on frit, then dried under a stream of nitrogen for 30 min resulting in 215 mg of white solid product. The XRPD pattern for (2R,5S)-7-Methyl-2-(4-methyl-6-(4-(trifluoromethyl)phenyl)pyrimidin-2-yl)-1,7-diazaspiro[4.4]nonan-6-one saccharinate is shown in FIG. 7.

### Example 6

Preparation of (2R,5S)-7-methyl-2-(4-methyl-6-(4-(trifluoromethyl)phenyl)pyrimidin-2-yl)-1,7-diazaspiro[4.4]nonan-6-one oxalate: 403 mg of (2R,5S)-7-Methyl-2-(4-methyl-6-(4-(trifluoromethyl)phenyl)pyrimidin-2-yl)-1,7-diazaspiro[4.4]nonan-6-one freebase was dissolved in 4.03 mL EtOH. 1.000 mL of this solution was added to a 4 mL vial. 23.8 mg of oxalic acid was dissolved in 1.000 mL of EtOH and added dropwise to the stirring (2R,5S)-7-Methyl-2-(4-methyl-6-(4-(trifluoromethyl)phenyl)pyrimidin-2-yl)-1,7-diazaspiro[4.4]nonan-6-one freebase solution. After 5 min, a white precipitate was evident and 2.000 mL of EtOH was added to the slurry to aid stirring. The resulting suspension was stirred overnight. The following day the suspension was filtered and dried on a frit under vacuum for 10 min yielding 106 mg of white solid. The XRPD pattern for (2R,5S)-7-Methyl-2-(4-methyl-6-(4-(trifluoromethyl)phenyl)pyrimidin-2-yl)-1,7-diazaspiro[4.4]nonan-6-one oxalate is shown in FIG. 8.

### Example 7

The single crystal structural information and refinement parameters for (2R,5S)-7-Methyl-2-(4-methyl-6-(4-(trifluoromethyl)phenyl)pyrimidin-2-yl)-1,7-diazaspiro[4.4]nonan-6-one hydrosulfate are shown in Table 1.

TABLE 1

| Empirical formula | C₂₀H₂₃F₃N₄O₅S |
|---|---|
| M/g · mol⁻¹ | 448.48 |
| T/K | 173(2) |
| Color | Colorless |
| Crystal system | Monoclinic |
| Space group | C2 |
| a/Å | 24.8814(11) |
| b/Å | 7.0450(2) |
| c/Å | 14.1375(6) |
| β/° | 119.032(3) |
| U/Å³ | 2166.77(16) |
| Z/Z′ | 4/1 |
| Dc/g cm⁻³ | 1.497 |
| μ/mm⁻¹ | 1.936 |
| Crystal size/mm | 0.34 × 0.06 × 0.02 |
| Reflections collected | 8294 |
| R(int) | 0.0626 |
| Data/restraints/parameters | 2760/1/309 |
| R1 [I > 2 (I)] | 0.560 |
| wR2 (all data) | 0.1226 |
| Largest peak, hole/e Å⁻³ | 0.363, −0.264 |

The most prominent XRPD diffraction peaks were (2θ): 7.8 ± 0.2°, 8.1 ± 0.2°, 12.6 ± 0.2°, 14.3 ± 0.2°, 16.5 ± 0.2°, 18.5 ± 0.2°, 19.6 ± 0.2°, 24.8 ± 0.2° and 25.3 ± 0.2°.

1-2. (canceled)

3. A pharmaceutically acceptable salt of a compound of formula (Ia): 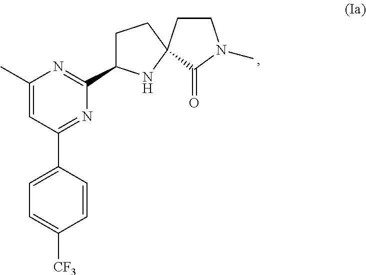
wherein the pharmaceutically acceptable salt is a citric acid (citrate) salt, a methanesulfonic acid (mesylate) salt, a sulfuric acid (hydrosulfate) salt, a saccharin (saccharinate) salt or an oxalic acid (oxalate) salt.

4. A crystalline form of the pharmaceutically acceptable salt of claim 3, wherein the pharmaceutically acceptable salt is (2R,5S)-7-Methyl-2-[4-methyl-6-[4-(trifluoromethyl)-phenyl]pyrimidin-2-yl]-1,7-diazaspiro[4.4]nonan-6-one citric acid (citrate) salt (E1).

5. (canceled)

6. The crystalline form of claim 4, having an XRPD pattern with peaks at 2θ values 15.2±0.2°, 23.7±0.2° and 24.8±0.2°.

7. (canceled)

8. The crystalline form of claim 6, having an XRPD pattern substantially as shown in FIG. 1.

9. A crystalline form of the pharmaceutically acceptable salt of claim 3, wherein the pharmaceutically acceptable salt is (2R,5S)-7-Methyl-2-[4-methyl-6-[4-(trifluoromethyl)-phenyl]pyrimidin-2-yl]-1,7-diazaspiro[4.4]nonan-6-one methanesulfonic acid (mesylate) salt (E2).

10. (canceled)

11. The crystalline form of claim 9, having an XRPD pattern with peaks at 2θ values 17.9±0.2°, 24.5±0.2° and 26.3±0.2°.

12. (canceled)

13. The crystalline form of claim 11, having an XRPD pattern substantially as shown in FIG. 3.

14. A crystalline form of the pharmaceutically acceptable salt of claim 3, wherein the pharmaceutically acceptable salt is (2R,5S)-7-Methyl-2-[4-methyl-6-[4-(trifluoromethyl)-phenyl]pyrimidin-2-yl]-1,7-diazaspiro[4.4]nonan-6-one sulfuric acid (hydrosulfate) salt (E3).

15. (canceled)

16. The crystalline form of claim 14, having an XRPD pattern with peaks at four or more 2θ values chosen from 8.1±0.2°, 12.6±0.2°, 14.3±0.2°, 16.5±0.2°, 18.5±0.2°, and 24.8±0.2°.

17. (canceled)

18. The crystalline form of claim 16, having an XRPD pattern substantially as shown in FIG. 5B.

19-23. (canceled)

24. A crystalline form of the pharmaceutically acceptable salt of claim 3, wherein the pharmaceutically acceptable salt is (2R,5S)-7-Methyl-2-[4-methyl-6-[4-(trifluoromethyl)-phenyl]pyrimidin-2-yl]-1,7-diazaspiro[4.4]nonan-6-one saccharin (saccharinate) salt (E5).

25. (canceled)

26. The crystalline form of claim 24, having 2θ values 6.4±0.2°, 12.8±0.2° and 15.4±0.2°.

27. (canceled)

28. The crystalline form of claim 26, having an XRPD pattern substantially as shown in FIG. 7.

29. A crystalline form of the pharmaceutically acceptable salt of claim 3, wherein the pharmaceutically acceptable salt is (2R,5S)-7-Methyl-2-[4-methyl-6-[4-(trifluoromethyl)-phenyl]pyrimidin-2-yl]-1,7-diazaspiro[4.4]nonan-6-one oxalic acid (oxalate) salt (E6).

30. (canceled)

31. The crystalline form of claim 29, having an XRPD pattern with peaks at 2θ values 7.9±0.2°, 16.0±0.2° and 16.7±0.2°.

32. (canceled)

33. The crystalline form of claim 31, having an XRPD pattern substantially as shown in FIG. 8.

34. (canceled)

35. A pharmaceutical composition comprising the pharmaceutically acceptable salt of claim 3 and one or more pharmaceutically acceptable carrier(s), diluents(s) and/or excipient(s).

36-38. (canceled)

39. A method of treating a disease or condition mediated by modulation of voltage-gated sodium channels, comprising administering the pharmaceutically acceptable salt of claim 3 to a subject in need thereof.

40-41. (canceled)

42. A crystalline form of (2R,5S)-7-Methyl-2-[4-methyl-6-[4-(trifluoromethyl)-phenyl]pyrimidin-2-yl]-1,7-diazaspiro[4.4]nonan-6-one (E4).

43. The crystalline form of claim 42, having an XRPD pattern with peaks at 2θ values 4.1±0.2°, 17.0±0.2°, and 22.5±0.2°.

44. The crystalline form of claim 43, having an XRPD pattern with peaks at 2θ values 4.1±0.2°, 12.5±0.2°, 14.9±0.2°, 17.0±0.2°, 20.8±0.2° and 22.5±0.2°.

45. The crystalline form of claim 44, having an XRPD pattern substantially as shown in FIG. 6.
